(19) United States
(12) Patent Application Publication
(10) Pub. No.: US 2024/0024393 A1
(43) Pub. Date: Jan. 25, 2024

(54) PHARMACEUTICAL COMPOSITION FOR PREVENTING OR TREATING CANCER, COMPRISING SQUIRREL FIBROMA VIRUS AND REOVIRUS

(71) Applicant: VIROCURE, INC., Seoul (KR)

(72) Inventors: Man-Bok KIM (deceased), Seoul (KR); Jin Ah CHO, Daejeon (KR); Ki-Hoon SONG, Seoul (KR)

(73) Assignee: VIROCURE, INC., Seoul (KR)

(21) Appl. No.: 17/757,251

(22) PCT Filed: Jan. 22, 2020
(86) PCT No.: PCT/KR2020/001053

§ 371 Date: Jan. 5, 2023

(30) Foreign Application Priority Data
Jan. 25, 2019 (KR) 10-2019-0009763
Dec. 31, 2019 (KR) 10-2019-0179160

(51) Int. Cl. A61K 35/765; C12N 15/86; A61K 45/06; A61P 35/00

(52) U.S. Cl. CPC A61K 35/765; C12N 15/86; A61K 45/06; A61P 35/00

(57) ABSTRACT

The present invention relates to a use of squirrel fibroma virus and a reovirus for preventing or treating cancer. A pharmaceutical composition comprising the squirrel fibroma virus and the reovirus, or a biological sample treated with the two viruses according to the present invention does not affect human normal cells but exhibits oncolytic activity by specifically infecting cancer cells, resulting in the inhibition of cancer cell growth.

### TECHNICAL FIELD

The present invention relates to a use of squirrel fibroma virus and reovirus for preventing or treating cancer.

### BACKGROUND ART

The incidence of cancer is continuously increasing, compared to the past, due to industrial development, changes in the global ecosystem and dietary life, etc., and research on anticancer drugs continues to grow.

Pharmaceuticals currently used can be divided into chemotherapeutic agents and biological agents, and biological anticancer agents using genes, enzymes, vaccines and viruses are still insufficient at the stage of commercialization, and the problem of chemical agents having various side effects caused by pharmacological action and toxicity is pointed out.

Accordingly, although various treatment methods such as surgery, radiation therapy and chemotherapy have been studied, a complete cure for cancer has not yet been achieved. Among these methods, since surgical therapy and radiation therapy are useful methods in the early stage of cancer development, for cancers that are difficult to detect in an early stage, the dependence on chemotherapy is gradually increasing. Chemotherapy has a history of more than 50 years, and hundreds of anticancer agents have been developed to date and used in clinical practice, but there are still few cases that can obtain clinically satisfactory effects.

Viruses are one of the biotherapeutics, and have the concept of targeted therapeutics that attack using genetic mutations in tumor cells. Oncolytic viruses selectively proliferate in cancer cells and induce tumor necrosis and death.

Oncolytic viruses are viruses used in cancer treatment, and anticancer therapy using them is called oncolytic viral therapy (OV therapy). Cancer treatment research using a wild-type oncolytic virus is distinguished from gene therapy which mainly sees an oncolytic effect by the expression of a therapeutic gene by inserting the conventional therapeutic gene into the virus, and the research on a wild-type oncolytic virus began when it became known that some wild-type viruses have inherently potent oncolytic ability.

It has been reported for a long time that cancer is cured naturally due to natural infection by various types of viruses, and since the research on the tumor-specific lytic mechanism by a wild-type virus started in earnest, cancer treatment research using a wild-type reovirus has reached the application to a phase 3 clinical trial. In addition, adenovirus, poliovirus, herpes simplex virus, and vesicular stomatitis virus have been developed, and methods to increase the efficiency and stability of viruses are being studied.

### DISCLOSURE

### Technical Problem

Therefore, while continuing research to find effective oncolytic viruses, the present inventors confirmed that, when squirrel fibroma virus is used in combination with a reovirus, due to host specificity, they not only exhibit high stability in infecting only cancer cells, not human normal cells, and tumoricidal ability, but also have a very excellent synergistic effect, compared to individual viral therapy, and the present invention was completed.

Accordingly, the present invention is directed to providing a pharmaceutical composition for preventing or treating cancer.

However, technical problems to be solved in the present invention are not limited to the above-described problems, and other problems which are not described herein will be fully understood by those of ordinary skill in the art from the following description.

### Technical Solution

To achieve the purposes of the present invention, the present invention provides a pharmaceutical composition for preventing or treating cancer, which comprises (a) squirrel fibroma virus and a reovirus; or (b) a biological sample treated with (a) as an active ingredient.

In addition, the present invention provides a method of preventing or treating cancer, which comprises administering (a) squirrel fibroma virus and a reovirus; or (b) a biological sample treated with (a) into a subject.

In addition, the present invention provides a use of (a) squirrel fibroma virus and a reovirus; or (b) a biological sample treated with (a) for preventing, alleviating or treating cancer.

Further, the present invention provides a use of (a) squirrel fibroma virus and a reovirus, or (b) a biological sample treated with (a) for preparing a preparation for preventing or treating cancer.

In one embodiment of the present invention, the cancer may be selected from the group consisting of gastric cancer, glioma, lung cancer, liver cancer, melanoma, prostate cancer, blood cancer, breast cancer, colorectal cancer, pancreatic cancer, brain cancer, ovarian cancer, and a combination thereof, but the present invention is not limited thereto.

In another embodiment of the present invention, the squirrel fibroma virus or reovirus may comprise a heterologous gene for treating cancer, but the present invention is not limited thereto.

In still another embodiment of the present invention, the heterologous gene for treating cancer may be inserted into an essential region or non-essential region in a squirrel fibroma virus gene or reovirus gene to increase anticancer activity, but the present invention is not limited thereto.

In yet another embodiment of the present invention, the squirrel fibroma virus or reovirus may be loaded in virus carrier cells and administered into the body, but the present invention is not limited thereto.

In yet another embodiment of the present invention, the squirrel fibroma virus may be comprised at 10² to 10¹³ PFU, but the present invention is not limited thereto.

In yet another embodiment of the present invention, the reovirus may be comprised at 10² to 10¹⁵ PFU, but the present invention is not limited thereto.

In yet another embodiment of the present invention, a titer ratio of the squirrel fibroma virus and reovirus may be 1:1 to 1000:1 (reovirus:squirrel fibroma virus), but the present invention is not limited thereto.

In yet another embodiment of the present invention, a titer ratio of the squirrel fibroma virus and reovirus may be 1:1 to 1:1000 (reovirus:squirrel fibroma virus), but the present invention is not limited thereto.

In yet another embodiment of the present invention, the biological sample may be prepared to kill a plurality of cancer cells by applying an effective amount of squirrel fibroma virus and reovirus to the biological sample ex vivo, but the present invention is not limited thereto.

In yet another embodiment of the present invention, the biological sample may be a bone marrow sample, an adipose-derived stem cell sample or a blood sample, but the present invention is not limited thereto.

In yet another embodiment of the present invention, the squirrel fibroma virus may be administered with a reovirus simultaneously, separately or sequentially, but the present invention is not limited thereto.

In yet another embodiment of the present invention, the squirrel fibroma virus or reovirus may be obtained using baby hamster kidney (BHK) cells, but the present invention is not limited thereto.

In yet another embodiment of the present invention, the pharmaceutical composition may further comprise a chemotherapeutic agent or immunotherapeutic agent, but the present invention is not limited thereto.

### Advantageous Effects

A pharmaceutical composition comprising squirrel fibroma virus and a reovirus, or a biological sample treated with the two types of viruses according to the present invention has the effect of inhibiting the growth of cancer cells by exhibiting oncolytic activity by specifically infecting cancer cells without affecting normal human cells.

In addition, since the combination of squirrel fibroma virus and a reovirus exhibits a very excellent synergistic effect compared to the individual viral therapy, the present invention can be effectively used in prevention and treatment of cancer.

### DESCRIPTION OF DRAWINGS

FIGS. 1A, 1B and 1C show the synergistic oncolytic effect of the combination of squirrel fibroma virus and a reovirus in gastric cancer cells, and provide graphs of confirming cancer cell death in the infection of gastric cancer cells (MKN28, AGS, and SNU668) without treatment (Mock), with a reovirus alone (REO), squirrel fibroma virus alone (SFV) and the combination of the reovirus and squirrel fibroma virus (REO+SFV) by CPE assay and WST assay, respectively.

FIG. 2 shows the synergistic oncolytic effect of the combination of squirrel fibroma virus and a reovirus in glioma cells, and provides graphs of confirming cancer cell death in the infection of glioma cells (U87MG and SNU489) without treatment (Mock), with a reovirus alone (REO), squirrel fibroma virus alone (SFV) and the combination of the reovirus and squirrel fibroma virus (REO+SFV) by CPE assay and WST assay.

FIG. 3 shows the synergistic oncolytic effect of the combination of squirrel fibroma virus and a reovirus in lung cancer cells, and provides a graph of confirming cancer cell death in the infection of lung cancer cells (A549) without treatment (Mock), with a reovirus alone (REO), squirrel fibroma virus alone (SFV) and the combination of the reovirus and squirrel fibroma virus (REO+SFV) by CPE assay and WST assay.

FIG. 4 shows the synergistic oncolytic effect of the combination of squirrel fibroma virus and a reovirus in liver cancer cells, and provides a graph of confirming cancer cell death in the infection of liver cancer cells (Hep3B) without treatment (Mock), with a reovirus alone (REO), squirrel fibroma virus alone (SFV), and the combination of the reovirus and squirrel fibroma virus (REO+SFV) by CPE assay and WST assay.

FIG. 5 shows the synergistic oncolytic effect of the combination of squirrel fibroma virus and reovirus in melanoma in vivo, and provides a graph obtained by measuring the change in tumor size after the infection of B16F10 melanoma allograft-induced C57BL/6 female mice with a reovirus alone (REO), squirrel fibroma virus alone (SFV), and the combination of the reovirus and squirrel fibroma virus (REO+SFV).

### MODES OF THE INVENTION

The present invention provides a pharmaceutical composition for preventing or treating cancer, which comprises (a) squirrel fibroma virus and a reovirus; or (b) a biological sample treated with (a) as an active ingredient.

According to one aspect of the present invention, the present invention provides a method of preventing or treating cancer, which comprises administering the (a) or (b) into a subject in need.

According to another aspect of the present invention, the present invention provides a use of the (a) or (b) for preventing, alleviating or treating cancer.

According to still another aspect of the present invention, the present invention provides a use of the (a) or (b) for preparing a preparation for preventing or treating cancer.

While continuing research to find effective tumoricidal (oncolytic) viruses, the present inventors confirmed that, when squirrel fibroma virus is used in combination with a reovirus, due to host specificity, they not only exhibit high stability in infecting only cancer cells, not human normal cells, and tumoricidal ability, but also have a very excellent synergistic effect, compared to individual viral therapy (see Examples 1 to 5).

The term “squirrel fibroma virus” used herein is isolated from an American gray squirrel as a host, belongs to Chordopoxvirinae in the family Poxviridae, and classified as Parapoxvirus sp. The squirrel fibroma virus of the present invention may be isolated from the above-mentioned host, and may be isolated by cell culture.

The squirrel fibroma virus of the present invention may be obtained using known technology such as in vivo passage using squirrels/woodchucks, primary squirrel kidney cells, or sheep choroid plexus cells.

In the present invention, the squirrel fibroma virus may be wild-type squirrel fibroma virus or an attenuated squirrel fibroma virus, but the present invention is not limited thereto.

The term “reovirus” used herein is a virus having a double-stranded segmented RNA genome, and refers to any virus classified in the family Reoviridae. The virion of the reovirus is 60 to 80 nm in diameter and has two concentric capsid shells. This genome consists of double-stranded RNA, which is 10 to 12 discontinuous segments, and has a total genome size of 16 to 27 kbp, and the RNA segments have different sizes.

In the present invention, the reovirus comprises not only a naturally occurring reovirus, but also a modified or recombinant reovirus. The reovirus is said to be “naturally occurring” when it can be isolated from nature and has not been artificially altered by humans. For example, the reovirus may be derived from a “field source,” that is, a person infected with a reovirus.

Although the reovirus may be modified, mammalian cells having an activated ras pathway may be lytically infected with the reovirus. In addition, the reovirus may be pretreated chemically or biochemically (e.g., by treatment with a protease such as chymotrypsin or trypsin) prior to the administration to proliferating cells. By pretreatment using a protease, the outer membrane or capsid of the virus may be removed, thereby increasing the infectivity of the virus. The reovirus may be coated with liposomes or micelles, and for example, to generate new infectious subviral particles, virions may be treated with chymotrypsin in the presence of an alkyl sulfate detergent at a micelle-forming concentration.

In the present invention, the reovirus may be a wild-type reovirus or an attenuated reovirus, but the present invention is not limited thereto.

The attenuated reovirus comprises an infectious, replicable reovirus virion in which a reovirus sigma 1 capsid protein, capable of being defected by the genome of wild-type reovirus S1 gene-deficient reovirus, is deficient. As such, the attenuated reovirus stems from the surprising observation that mutated reoviruses in which a detectable reovirus sigma 1 capsid protein is deficient preferentially avoid the cytopathic effect on non-malignant cells while unexpectedly maintaining the ability to productively infect target tumor cells. As described above, prior to the present disclosure, particles of a sigma-1 deficient reovirus were understood to be non-infectious.

In a specific embodiment, the attenuated reovirus may comprise the S4 gene of a mutated reovirus. The reovirus wild-type S4 gene encodes a reovirus capsid sigma-3 polypeptide involved in virion processing during the replicative infection of host cells with reoviruses.

In a specific embodiment, the attenuated reovirus may comprise a mutated reovirus S4 gene comprising one or more mutations in a genomic sequence encoding the reovirus sigma-3 polypeptide compared to the wild-type S4 gene sequence.

The attenuated reovirus lacks a detectable sigma-1 capsid protein, but is unexpectedly infectious. As described above, it was seen that sigma-1 is involved in the binding and attachment of a reovirus to cells via a cell surface sialic acid residue in the initial stage of viral replication infection. Despite lacking detectable sigma-1, the attenuated reovirus described herein is capable of host cell entry and replication of cytolytic viruses. In addition, the attenuated reovirus shows the surprising characteristic of inducing decreased levels (i.e., reduced statistical significance) of one or more cytopathic effects on non-malignant cells compared to the level of the cytopathic effect exhibited on non-malignant cells by naturally-occurring non-attenuated reoviruses.

The attenuated reovirus may be derived from any reovirus, meaning a member of the family Reoviridae and include reoviruses with various types of affinity, which may be obtained from a variety of sources.

In a specific embodiment, the reovirus may be a mammalian reovirus, and in another embodiment, a human reovirus. In another embodiment (e.g., to be used in an animal model having relevance to human disease or to be used in related veterinary applications), an attenuated reovirus may be derived from one or more reoviruses exhibiting affinity to cells of different mammalian species, including non-human primates (e.g., a chimpanzee, a gorilla, a macaque, a monkey, etc.), rodents (e.g., a mouse, a rat, a gerbil, a hamster, a rabbit, a guinea pig, etc.), dogs, cats, common livestock (e.g., cattle, a horse, a pig, a goat), or alternatively, a reovirus (e.g., avian reovirus) having distinct affinity may be used.

Attenuated reoviruses may be derived by the generation and identification of sigma-1-deficient and/or sigma-1-defective mutant(s) according to molecular biological approaches (in a specific embodiment, additionally or alternatively, also including the generation and identification of sigma 3-deficient and/or sigma 3-defective mutant(s)), and other methodology including the isolation of naturally occurring sigma-1-deficient and/or sigma-1-defective mutant(s) and/or sigma 3 mutant(s), and/or artificial induction of such sigma-1 (and/or sigma-3) mutants by chemical, physical and/or genetic techniques (e.g., selective recombination of reovirus genes in productively-infected host cells).

The attenuated reoviruses comprise infectious, replicable reovirus virions (that is, viral particles comprising a viral genome, a core protein and a protein coat) that lack the wild-type reovirus S1 gene and consequently lack a detectable reovirus sigma-1 capsid protein.

In a specific embodiment, the attenuated reoviruses lack the wild-type reovirus S4 gene and express a mutated reovirus sigma-3 capsid protein. As known in the related art, an infectious, replicable reovirus is one that can be internalized by binding to host cells upon introduction into suitable host cells under appropriate conditions for a sufficient time and directs the replication of the reovirus genome and the biosynthesis of a reovirus structural protein in a manner that, upon subsequent release from host cells, allows the assembly of complete progeny reoviruses capable of infecting other host cells productively to perpetuate the replication cycle of the virus.

Attenuated reoviruses for cancer treatment are disclosed in, for example, US Unexamined Patent Application Nos. US2009/0214479 and US2009/0104162, each of which is incorporated herein by reference in its entirety.

The human reovirus may consist of three serotypes as follows: type 1 Lang (T1L), type 2 Jones (T2J), and type 3 Dearing (T3D) or type 3 Abney (T3A). The three serotypes may be easily isolated based on a neutralization reaction and hemagglutinin inhibition assay.

In the present invention, the squirrel fibroma virus or reovirus may be obtained using baby hamster kidney (BHK) cells (e.g., BHK-21 cells), but the present invention is not limited thereto.

In the term “BHK-21 cell” used herein, BHK is an abbreviation of Baby Hamster Kidney, and BHK cells were originally isolated by polyoma transformation of hamster cells, and can be used as a substrate for viral propagation with respect to a vaccine and virus-mediated expression. In addition, BHK cells are useful as a host cell line for stable expression of various recombinant proteins. The BHK Strain 21 (BHK-21) cell line was derived from the baby hamster kidneys of five unsexed, 1-day-old hamsters, which were identified by IA Macpherson and MGP Stoker in March 1961. The hamsters were used to generate BHK-21 cells, and is generally known as the Syrian or golden hamster (Mesocricetus auratus).

In the term “biological sample treated with (a)” used herein, the “biological sample” refers to any biological sample obtained from a subject, a cell line, a tissue culture or other sources of cells, such as adult stem cells (adipose-derived stem cells, bone marrow stem cells) or cord blood stem cells. Methods for obtaining a tissue biopsy and a body fluid from a mammal are well known in the art. For example, adipose-derived stem cells may be pretreated with oncolytic viruses and administered to cancer patients.

In the present invention, the biological sample may be prepared to kill a plurality of cancer cells by applying effective amounts of squirrel fibroma virus and a reovirus to an ex vivo biological sample, but the present invention is not limited thereto.

In the present invention, the biological sample may be a bone marrow sample, an adipose-derived stem cell sample, or a blood sample, but the present invention is not limited thereto.

In the present invention, the squirrel fibroma virus or reovirus may comprise a heterologous gene for cancer treatment, but the present invention is not limited thereto.

The term “heterologous gene” used herein is used to mean the acceptance of any gene that is not found in a viral genome. The heterologous gene may be an allelic variant of a wild-type gene or a mutant gene.

The heterologous gene for cancer treatment may be inserted into the essential region or non-essential region in a squirrel fibroma virus gene or reovirus gene to increase anticancer activity.

The heterologous gene may be operably linked to a control sequence that expresses a heterologous gene in a cell under an in vivo condition. Therefore, the virus of the present invention may be used to deliver heterologous gene/genes to cells under an in vivo condition that can express the heterologous gene(s). The gene usually encodes a protein that can improve the oncolytic property of the virus. The gene may encode a cytotoxin protein or a protein that can promote/improve an antitumor immune response.

A heterologous gene includes all genes that are inserted into a replicable oncolytic virus or an existing non-replicable viral vector to treat cancer.

The term “therapeutic gene” used herein is considered to describe all of the various genes whose expression brings preferable results, that is, exhibit an anticancer effect. The squirrel fibroma virus or reovirus of the present invention may include one or more desired sequences encoding the therapeutic gene. The therapeutic gene may have pharmacological or prophylactic activity when appropriately administered to a patient, particularly, a patient suffering from a disease or condition or a patient to be protected from such a disease or condition.

The pharmacological or prophylactic activity is meant to be expected to be associated with a beneficial effect on the course or symptom of the disease or condition. The desired sequence may originate from a cell of the same type as the target cell into which it is introduced or a different type therefrom, and encodes a polypeptide, particularly, all or a part of a therapeutic or prophylactic polypeptide exhibiting a therapeutic or prophylactic property. The polypeptide is understood to be any translational product of a polynucleotide, regardless of size and glycosylation, and includes a peptide and a protein. A therapeutic polypeptide includes polypeptides that can compensate for a deleted or deficient protein in an animal or human organism or those that act through a toxic effect to limit or remove harmful cells from the body. These may also be immunity-conferring polypeptides that act as endogenous antigens to induce either humoral or cellular responses, or both, and include, for example, a drug-sensitizing gene, a proapoptotic gene, a cytostatic gene, a cytotoxic gene, a tumor suppressor gene, an antigenic gene, an anti-angiogenic gene, a cytokine gene, and the like, but the present invention is not limited thereto.

The drug-sensitizing gene is a gene for an enzyme converting a non-toxic prodrug into a toxic material and is also called a suicide gene since the gene-introduced cells are killed. That is, when a prodrug that is non-toxic to normal cells is systemically administered, only in cancer cells is the prodrug converted into a toxic metabolite and the sensitivity to a drug is changed, thereby destroying cancer cells. Representative examples of the drug-sensitizing genes include Herpes simplex virus-thymidine kinase (HSVtk) gene and ganciclovir, and E. coli cytosine deaminase (CD) gene and 5-fluorocytosine (5-FC).

The proapoptotic gene refers to a nucleotide sequence that induces programmed cell death when expressed. Proapoptotic genes known by those of ordinary skill in the art include p53, adenovirus E3-11.6K (derived from Ad2 and Ad5) or adenovirus E3-10.5K (derived from Ad), an adenovirus E4 gene, a p53 pathway gene and a caspase-encoding gene.

The cytostatic gene refers to a nucleotide sequence that is expressed in a cell and stops the cell cycle during the cell cycle. Representative examples of the cytostatic genes include p21, retinoblastoma genes, E2F-Rb fusion protein genes, genes encoding a cyclin-dependent kinase inhibitor (e.g., p16, p15, p18 and p19), and growth arrest specific homeobox (GAX) genes.

The cytotoxic gene refers to a nucleotide sequence expressed in a cell and exhibiting a toxic effect. Examples of the cytotoxic genes include nucleotide sequences encoding pseudomonas exotoxin, ricin toxin, and diphtheria toxin.

The tumor suppressor gene refers to a nucleotide sequence that is expressed in a target cell to suppress a tumor phenotype or induce cell death. Representative examples of the tumor suppressor genes include tumor necrosis factor-α (TNF-α), a p53 gene, an APC gene, a DPC-4/Smad4 gene, a BRCA-1 gene, a BRCA-2 gene, a WT-1 gene, a retinoblastoma gene (Lee et al., Nature, 329, 642, 1987), an MMAC-1 gene, an adenomatous polyposis coil protein, a deleted in colon cancer (DCC) gene, an MMSC-2 gene, an NF-1 gene, a nasopharyngeal carcinoma tumor suppressor gene that maps at chromosome 3p21.3, an MTS1 gene, a CDK4 gene, an NF-1 gene, an NF-2 gene, and a VHL gene.

The antigenic gene refers to a nucleotide sequence that is expressed in a target cell and produces a cell surface antigenic protein capable of being recognized by an immune system. Examples of the antigenic genes known by those of ordinary skill in the art include carcinoembryonic antigen (CEA) and p53 (Levine, A., PCT International Publication No. WO 94/02167).

More specific examples of polypeptides encoded by a therapeutic gene include cytokines (α, β or γ-interferons, interleukins, particularly, IL-2, IL-6, IL-10 and IL-12, tumor necrosis factors (TNFs), chemokines, colony-stimulatory factors, such as GM-CSF, C-CSF, and M-CSF), immunostimulatory polypeptides (B7.1, B7.2, etc.), clotting factors (FVIII and FIX), growth factors (transforming growth factor (TGF), fibroblast growth factor (FGF), etc.), enzymes (urease, renin, thrombin, and metalloproteinase), nitric oxide synthase (NOS, SOD, and catalase), enzyme inhibitors (alpha 1-antitrypsin, antithrombin III, viral protease inhibitors, plasminogen activator inhibitor PAI-1), a cystic fibrosis transmembrane conductance regulator (CFTR) protein, insulin, dystrophin, MHC class I or II antigen, a polypeptide capable of modulating/regulating the expression of a cellular gene, polypeptides capable of inhibiting bacterial, parasitic or viral infection or its occurrence (antigenic polypeptide, antigenic epitopes, transdominant variants competitively inhibiting the action of a natural protein), apoptosis inducers or inhibitors (Bax, Bcl2, BclX, etc.), cytostatic agents (p21, p16, and Rb), apolipoproteins (ApoAI, ApoAIV, ApoE, etc.), inhibitors of angiogenesis (angiostatin, endostatin), angiogenic polypeptides (the vascular endothelial growth factor (VEGF) family, FGF family, and CCN family including CTGF, Cyr61 and Nov), an oxygen radical scavenger, a polypeptide with an antitumor effect, genes encoding an antibody, a toxin, an immunotoxin and a marker (β-galactosidase or luciferase), or any other genes of interest recognized in the art as useful for the treatment or prevention of a clinical condition. Suitable antitumor genes include tumor suppressor genes (e.g., Rb, p53, DCC, NF-1, Wilm's tumor, NM23, BRUSH-1, p16, p21, p56, p73 and mutants thereof), a suicide gene product, an antibody, and those encoding a polypeptide inhibiting a cell division or transduction signal, but the present invention is not limited thereto. The suicide gene includes gene(s) encoding protein(s) having cytosine deaminase activity, thymidine kinase activity, uracil phosphoribosyl transferase activity, purine nucleoside phosphorylase activity and/or thymidylate kinase activity, but the present invention is not limited thereto. The therapeutic gene may be inserted into the essential region or non-essential region in a squirrel fibroma virus or reovirus gene to increase the anticancer activity thereof.

In the present invention, the squirrel fibroma virus or reovirus may be loaded in virus carrier cells (e.g., Molecular Therapy. 2009 Vol 17:1667-1676) and administered into the body, and thus its therapeutic effect in vivo may further increase.

Meanwhile, the present invention relates to a pharmaceutical composition for preventing or treating cancer, and a method of preventing or treating cancer.

The term “prevention” used herein means all actions of inhibiting cancer or delaying the development thereof by administration of the composition of the present invention.

The term “treatment” used herein refers to all actions involved in alleviating or beneficially changing cancer by administration of the composition of the present invention, and refers to the attempt to achieve useful or preferable results, including clinical results. Whether detectable or undetectable, useful or preferable clinical results may include the alleviation or amelioration of one or more symptoms or conditions, a reduction in the scope of a disease, the stabilization of a disease state, the inhibition of the occurrence of a disease, the inhibition of the spread of a disease, the delay or slowing of the occurrence of a disease, the amelioration or mitigation of a disease state, and (partial or total) deterioration, but the present invention is not necessarily limited thereto. In addition, the “treatment” may mean the extension of the life of a patient more than that predicted in the absence of treatment. In addition, the “treatment” may mean the inhibition of disease progression or temporary slowing the disease progression, and may be associated with the permanent cessation of disease progression.

Cancer diseases that can be a target for treatment in the present invention include hematological cancer, colorectal cancer, brain cancer, glioma, gliosarcoma, anaplastic astrocytoma, mediulloblastoma, gastric cancer, lung cancer, small cell lung carcinoma, cervical carcinoma, colon cancer, rectal cancer, chordoma, throat cancer, Kaposi's sarcoma, lymphangiosarcoma, lymphagioendotheliosarcoma, colorectal carcinoma, endometrial cancer, ovarian cancer, breast cancer, pancreatic cancer, prostate cancer, renal cell carcinoma, hepatocarcinoma, cholangiocarcinoma, choriocarcinoma, seminoma, testicular tumors, Wilms' tumors, Ewing's tumors, bladder carcinoma, hemangiosarcoma, endothelial sarcoma, adenocarcinoma, sweat gland carcinoma, sebaceous adenocarcinoma, papillary carcinoma, papillary adenosarcoma, cystic adenosarcoma, bronchial carcinoma, medullary carcinoma, mastocytoma, mesothelioma, synovioma, melanoma, myofibroma, rhabdomyoma, neuroblastoma, retinoblastoma, oligodendroglioma, acoustic neuroma, hemangioblastoma, meningioma, pinealoma, ependymoma, craniopharyngioma, epithelial carcinoma, embryonal carcinoma, epidermoid carcinoma, basal cell carcinoma, fibrosarcoma, myxoma, myxosarcoma, liposarcoma, chondrosarcoma, osteogenic sarcoma, and leukemia.

In the present invention, the cancer may be selected from the group consisting of gastric cancer, glioma, lung cancer, liver cancer, melanoma, prostate cancer, blood cancer, breast cancer, colorectal cancer, pancreatic cancer, brain cancer, ovarian cancer, and a combination thereof, but the present invention is not limited thereto.

The virus of the present invention may be administered in combination or administered in combination with another therapy such as chemotherapy, radiation therapy, or another anti-viral therapy. For example, the virus may be administered before or after the removal of primary tumors through surgery, or before, concurrently with or after treatment such as radiation therapy or conventional chemotherapy. The virus may be administered together or sequentially with other oncolytic viruses exhibiting specificity for various tumor cell types.

Therapies commonly used to cure, prevent or treat cancer comprise surgery, chemotherapy, radiation therapy, hormone therapy, biological therapy, and immunotherapy, but the present invention is not limited thereto.

In the present invention, the pharmaceutical composition may further comprise a chemotherapeutic agent or an immunotherapeutic agent, but the present invention is not limited thereto.

The term “administration” used herein means providing the composition of the present invention to a subject by a suitable method.

The term “subject” used herein refers to all animals such as a human, a monkey, a dog, a goat, a pig or a mouse with cancer capable of being alleviated by administration of the composition of the present invention.

The active ingredient of the present invention is administered to a subject at a pharmaceutically effective amount.

The term “pharmaceutically effective amount” used herein refers to an amount sufficient for treating a disease at a reasonable benefit or risk ratio applicable for medical treatment, and may be determined by a type of a subject's disease, severity, drug activity, sensitivity to a drug, administration time, an administration route and an excretion rate, the duration of treatment and drugs simultaneously used, and other parameters well known in the medical field.

The effective amount of the virus is a dose required for a sufficient time to alleviate, improve, mitigate, ameliorate, stabilize a disease, inhibit the spread of a disease, slow or delay the progression of a disease, and cure a disease. For example, the effective amount may be an amount sufficient for achieving the effect of reducing the number of cancer cells or destroying cancer cells, reducing the number of cells chronically infected with a virus or destroying the cells, or inhibiting the growth and/or proliferation of the above-described cells.

The effective amount may vary depending on numerous factors such as pharmacokinetic properties of a virus, an administration method, age, the health condition and body weight of a patient, the nature and range of a disease state, the number of treatments and the latest form of treatment, and may also depend on, for example, the virulence and titer of a virus. The appropriate amount may be adjusted by those of ordinary skill in the art. The virus may be initially administered in an appropriate dose as needed, depending on a patient's clinical response. The effective amount of viruses may be determined empirically and may depend on the maximum amount of viruses that can be safely administered and the minimum amount of viruses that cause a preferable result.

When the virus is administered systemically, to cause a clinical effect similar to that realized by injecting the virus into a diseased site, the administration of a considerably high dose of the virus may be required. However, proper dosage levels should be the minimum amounts for realizing a preferable result.

The concentration of the administered virus may vary depending on the virulence of a squirrel fibroma virus or reovirus strain to be administered and the characteristics of cells being targeted.

In the present invention, the squirrel fibroma virus may be comprised at 10² to 10¹³ plaque-forming unit (PFU). For example, the squirrel fibroma virus may be comprised at 10² to 10¹², 10² to 10¹¹, 10² to 10¹⁰, 10² to 10⁹, 10² to 10⁸ or 10² to 10⁷ PFU; 10³ to 10¹³, 10³ to 10¹², 10³ to 10¹¹, 10³ to 10¹⁰, 10³ to 10⁹, 10³ to 10⁸ or 10³ to 10⁷ PFU; 10⁴ to 10¹³, 10⁴ to 10¹², 10⁴ to 10¹¹, 10⁴ to 10¹⁰, 10⁴ to 10⁹, 10⁴ to 10⁸ or 10⁴ to 10⁷ PFU; or 10⁵ to 10¹³, 10⁵ to 10¹², 10⁵ to 10¹¹, 10⁵ to 10¹⁰, 10⁵ to 10⁹, 10⁵ to 10⁸ or 10⁵ to 10⁷ PFU.

In addition, in the present invention, the squirrel fibroma virus may be comprised at 0.01 to 10 multiplicity of infection (MOI). For example, the squirrel fibroma virus may be comprised at 0.01 to 9, 0.01 to 8, 0.01 to 7, 0.01 to 6 or 0.01 to 5 MOI; 0.05 to 10, 0.05 to 9, 0.05 to 8, 0.05 to 7, 0.05 to 6 or 0.05 to 5 MOI; or 0.1 to 10, 0.1 to 9, 0.1 to 8, 0.1 to 7, 0.1 to 6 or 0.1 to 5 MOI.

In the present invention, the reovirus may be comprised at 10² to 10¹⁵ PFU. For example, the reovirus may be comprised at 10² to 10¹⁴, 10² to 10¹³, 10² to 10¹², 10² to 10¹¹, 10² to 10¹⁰, 10² to 10⁹, 10² to 10⁸ or 10² to 10⁷ PFU; 10³ to 10¹⁵, 10³ to 10¹⁴, 10³ to 10¹³, 10³ to 10¹², 10³ to 10¹¹, 10³ to 10¹⁰, 10³ to 10⁹, 10³ to 10⁸ or 10³ to 10⁷ PFU; 10⁴ to 10¹⁵, 10⁴ to 10¹⁴, 10⁴ to 10¹³, 10⁴ to 10¹², 10⁴ to 10¹¹, 10⁴ to 10¹⁰, 10⁴ to 10⁹, 10⁴ to 10⁸ or 10⁴ to 10⁷ PFU; or 10⁵ to 10¹⁵, 10⁵ to 10¹⁴, 10⁵ to 10¹³, 10⁵ to 10¹², 10⁵ to 10¹¹, 10⁵ to 10¹⁰, 10⁵ to 10⁹, 10⁵ to 10⁸ or 10⁵ to 10⁷ PFU.

In addition, in the present invention, the reovirus may be comprised at 0.1 to 100 MOI. For example, the reovirus may be comprised at 0.2 to 100, 0.3 to 100, 0.4 to 100, 0.5 to 100, 0.6 to 100, 0.7 to 100, 0.8 to 100, 0.9 to 100, or 1 to 100 MOI.

In the present invention, the titer ratio of the squirrel fibroma virus and the reovirus may be 1:1 to 1000:1 (reovirus:squirrel fibroma virus), but the present invention is not limited thereto. For example, the titer ratio of the squirrel fibroma virus and the reovirus may be 1:1 to 900:1, 1:1 to 800:1, 1:1 to 700:1, 1:1 to 600:1, 1:1 to 500:1, 1:1 to 400:1, 1:1 to 300:1, 1:1 to 200:1, 1:1 to 100:1, 1:1 to 90:1, 1:1 to 80:1, 1:1 to 70:1, 1:1 to 60:1, 1:1 to 50:1, 1:1 to 40:1, 1:1 to 30:1, 1:1 to 20:1, or 1:1 to 10:1 (reovirus:squirrel fibroma virus).

In addition, in the present invention, the titer ratio of the squirrel fibroma virus and the reovirus may be 1:1 to 1000:1 (squirrel fibroma virus:reovirus), but the present invention is not limited thereto. For example, the titer ratio of the squirrel fibroma virus and the reovirus may be 1:1 to 900:1, 1:1 to 800:1, 1:1 to 700:1, 1:1 to 600:1, 1:1 to 500:1, 1:1 to 400:1, 1:1 to 300:1, 1:1 to 200:1, 1:1 to 100:1, 1:1 to 90:1, 1:1 to 80:1, 1:1 to 70:1, 1:1 to 60:1, 1:1 to 50:1, 1:1 to 40:1, 1:1 to 30:1, 1:1 to 20:1, or 1:1 to 10:1 (squirrel fibroma virus:reovirus).

The effective amount of the viruses may be repeatedly administered according to the effect of the initial treatment regimen. Generally, the administration is periodically administered during monitoring for all responses. It can be easily understood by those of ordinary skill in the art that, depending on the administration schedule and the selected route, a lower or higher dose than that indicated above may be administered.

In the present invention, the squirrel fibroma virus may be simultaneously, separately or sequentially administered with the reovirus, but the present invention is not limited thereto.

Meanwhile, the pharmaceutical composition according to the present invention may further comprise an appropriate carrier, excipient and/or diluent conventionally used, other than the active ingredient, to prepare a pharmaceutical composition. In addition, the pharmaceutical composition may be formed in an oral formulation such as a powder, a granule, a tablet, a capsule, a suspension, an emulsion, a syrup, or an aerosol, an external preparation, a suppository, or a sterile injectable solution.

When the composition is formulated, it can be prepared using generally used diluents or excipients such as a carrier, a stabilizer, an anti-bacterial agent, a buffer, a filler, a thickening agent, a binder, a wetting agent, a disintegrant, and a surfactant.

The pharmaceutical composition of the present invention may be administered into a subject by various routes. All methods for administration may be expected, and may include, for example, oral administration, intranasal administration, intratumoral administration, transbronchial administration, arterial injection, intravenous injection, subcutaneous injection, intramuscular injection or intraperitoneal injection. A daily dose may be administered once or in several divided portions a day.

Hereinafter, to help in understanding the present invention, exemplary examples will be suggested. However, the following examples are merely provided to more easily understand the present invention, and not to limit the present invention.

### EXAMPLES

### Example 1. Confirmation of Death of Gastric Cancer Cell Line

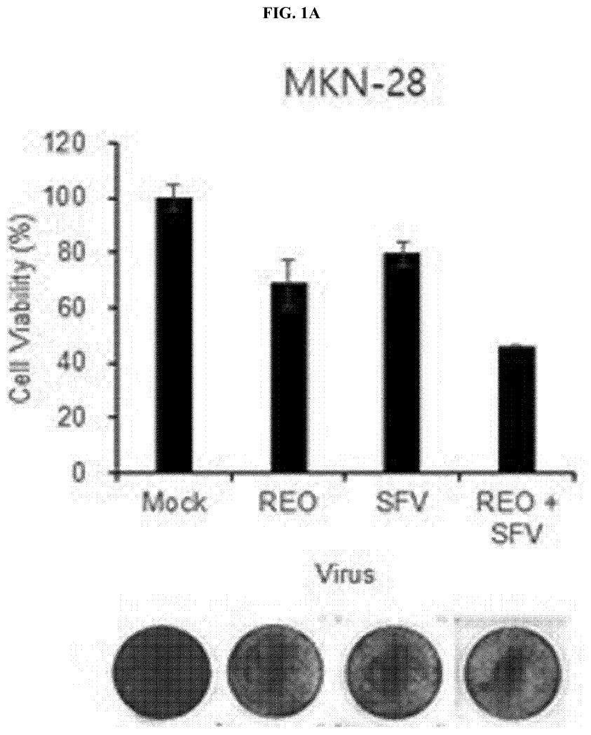

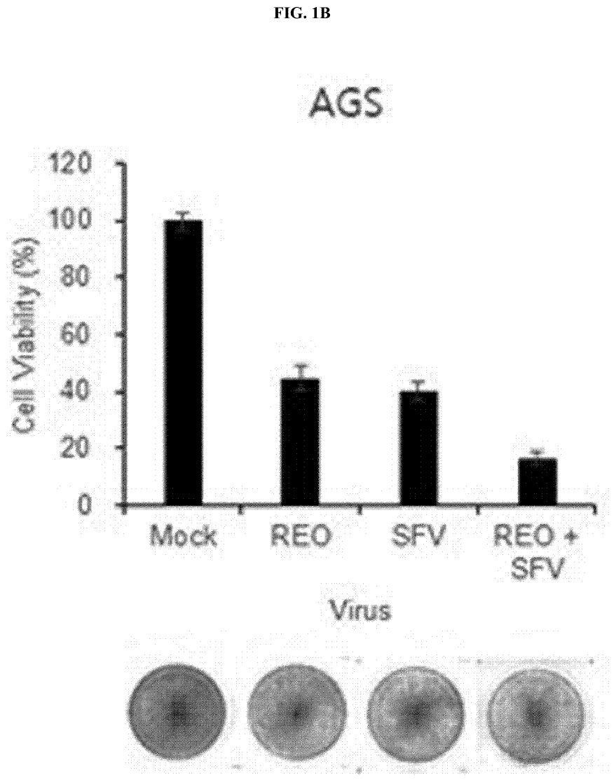

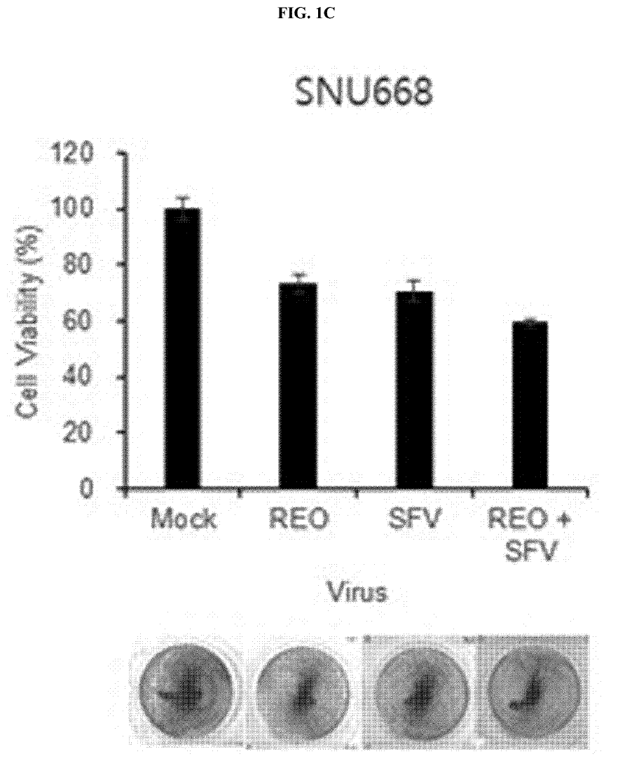

Gastric cancer cell lines (MKN28, AGS, and SNU668) were infected without treatment (Mock), with a wild-type reovirus alone (REO, MKN28 1 MOI; AGS and SNU668 100 MOI), wild-type squirrel fibroma virus alone (SFV, MKN28 0.32 MOI; AGS and SNU668 3.2 MOI), or the combination of the reovirus and squirrel fibroma virus (REO+SFV), and the death of cancer cells was confirmed by CPE assay. Specifically, the cancer cells were seeded in a 96-well plate and infected with the viruses, and then 4 days after infection, cell viability was measured by WST assay. The results converted to graphs are shown in FIGS. 1A, 1B and 1C.

The greater the anticancer effect, the less the number of cells stained with crystal violet. In the end, when the survival rate of cancer cells is greatly reduced due to the anticancer effect, it may be considered that the anticancer effect is large. As shown in FIGS. 1A, 1B and 1C, the relative cell viability was the lowest when infected with the combination of the reovirus and the squirrel fibroma virus, compared to when infected alone (the rightmost bar graph of each drawing).

The above results show that the combined use of a reovirus and squirrel fibroma virus exhibits a synergistic anticancer effect, showing the most excellent anticancer effect.

### Example 2. Confirmation of Death of Glioma Cell Line

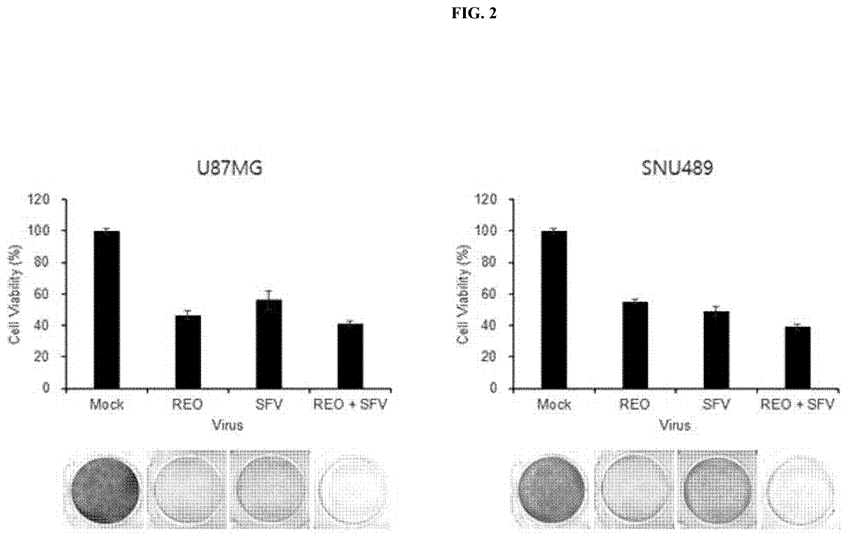

Glioma cell lines (U87MG and SNU489) were infected without treatment (Mock), with a wild-type reovirus alone (REO, 10 MOI), wild-type squirrel fibroma virus alone (SFV, U87MG 0.16 MOI; SNU489, 1.6 MOI) or the combination of the reovirus and squirrel fibroma virus (REO+SFV), and the death of cancer cells was confirmed by CPE assay. Specifically, the cancer cells were seeded in a 96-well plate and infected with the viruses, and then 4 days after infection, cell viability was measured by WST assay. The results converted to graphs are shown in FIG. 2.

As a result, as shown in FIG. 2, the relative cell viability was the lowest when infected with the combination of the reovirus and the squirrel fibroma virus, compared to when infected alone (the rightmost bar graph of FIG. 2).

The above results show that the combined use of a reovirus and squirrel fibroma virus exhibits a synergistic anticancer effect, showing the most excellent anticancer effect.

### Example 3. Confirmation of Death of Lung Cancer Cell Line

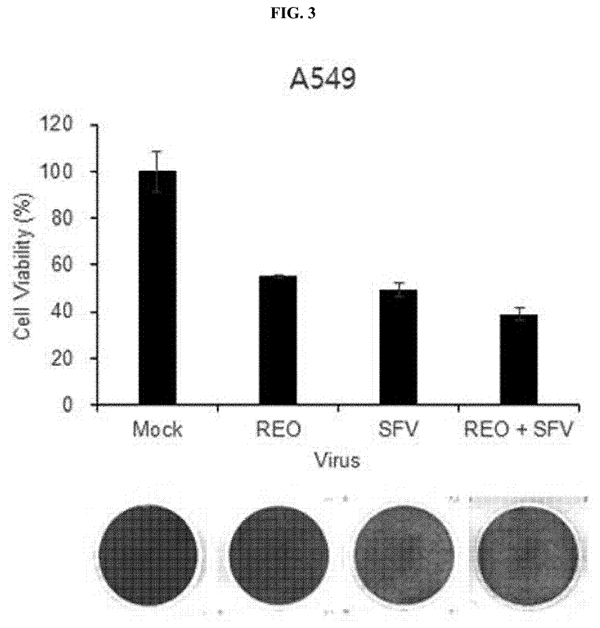

A lung cancer cell line (A549) was infected without treatment (Mock), with a wild-type reovirus alone (REO, 10 MOI), wild-type squirrel fibroma virus alone (SFV, 3.2 MOI) or the combination of the reovirus and squirrel fibroma virus (REO+SFV), and the death of cancer cells was confirmed by CPE assay. Specifically, the cancer cells were seeded in a 96-well plate and infected with the viruses, and then 4 days after infection, cell viability was measured by WST assay. The result converted to a graph is shown in FIG. 3.

As a result, as shown in FIG. 3, the relative cell viability was the lowest when infected with the combination of the reovirus and the squirrel fibroma virus, compared to when infected alone (the rightmost bar graph of FIG. 3).

The above results show that the combined use of a reovirus and squirrel fibroma virus exhibits a synergistic anticancer effect, showing the most excellent anticancer effect.

### Example 4. Confirmation of Death of Liver Cancer Cell Line

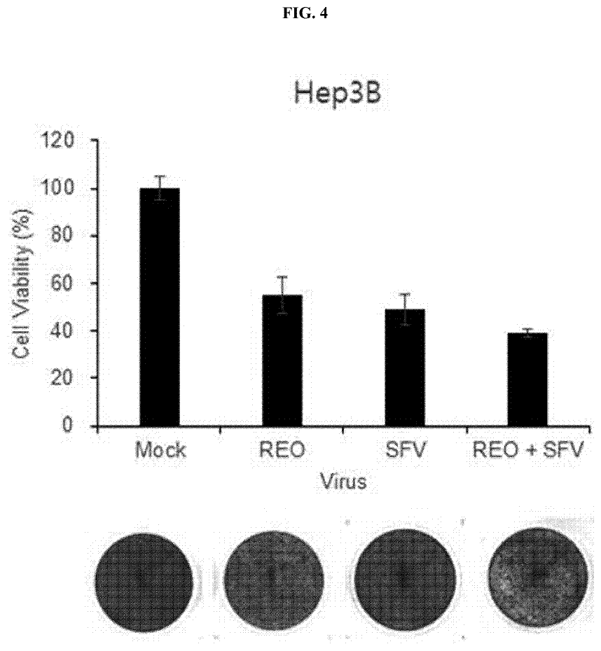

A liver cancer cell line (Hep3B) was infected without treatment (Mock), with a wild-type reovirus alone (REO, 1 MOI), wild-type squirrel fibroma virus alone (SFV, 0.32 MOI), or the combination of the reovirus and squirrel fibroma virus (REO+SFV), and the death of cancer cells was confirmed by CPE assay. Specifically, the cancer cells were seeded in a 96-well plate and infected with the viruses, and then 4 days after infection, cell viability was measured by WST assay. The result converted to a graph is shown in FIG. 4.

As a result, as shown in FIG. 4, the relative cell viability was the lowest when infected with the combination of the reovirus and the squirrel fibroma virus, compared to when infected alone (the rightmost bar graph of FIG. 4).

The above results show that the combined use of a reovirus and squirrel fibroma virus exhibits a synergistic anticancer effect, showing the most excellent anticancer effect.

### Example 5. Anticancer Effect on Melanoma

Tumor-induced allograft animal models were manufactured by injecting mouse melanoma cells (B16F10 cell) into C57BL/6 mice (female, DOB: 2019-, 5-week-old) at 1×10⁵ cells/mice. Afterward, the animal models were infected with a wild-type reovirus alone (1×10⁷ PFU/injection), wild-type squirrel fibroma virus alone (1×10⁴ PFU/injection) or the combination of the reovirus and squirrel fibroma virus, and then the death of cancer cells was confirmed with the decrease in tumor size. When the initial size of the tumor was 130 mm³, viral infection was carried out. The next day, viral infection was also carried out, and a tumor size was measured after the total of two viral injections.

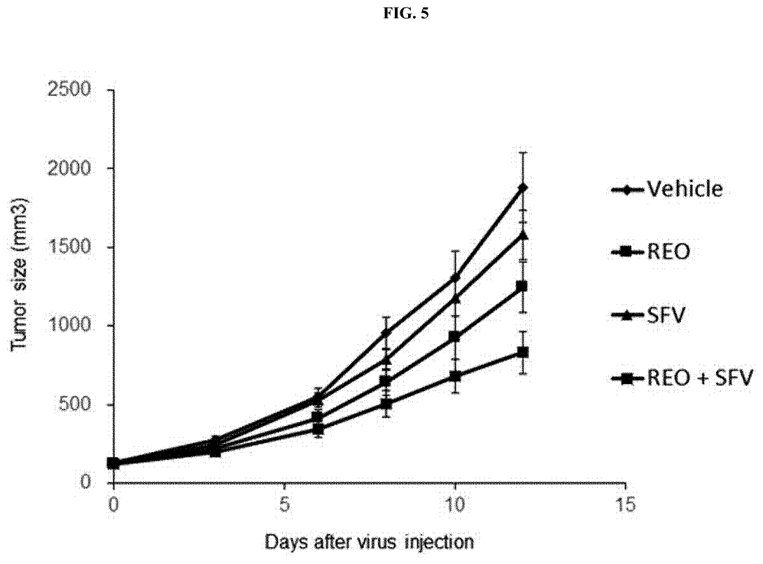

As a result, as shown in FIG. 5, compared to the negative control CsCl (Vehicle), when each type of virus was injected alone, the tumor size decreased, and particularly, when the combination of two types of viruses was injected, the tumor size decreased, that is, the anticancer effect (synergistic anticancer effect) significantly increased (FIG. 5).

The above results of Examples 1 to 5 show the highest anticancer effect in the case of the infection with the combination of viruses, compared with the infection with a reovirus or squirrel fibroma virus alone, and such a result shows that the combined use of a reovirus and squirrel fibroma virus exhibits the most excellent anticancer effect due to the synergistic anticancer effect. In addition, it shows that the combined use of the reovirus and the squirrel fibroma virus can exhibit an anticancer effect on various carcinomas.

It should be understood by those of ordinary skill in the art that the above description of the present invention is exemplary, and the exemplary embodiments disclosed herein can be easily modified into other specific forms without departing from the technical spirit or essential features of the present invention. Therefore, the exemplary embodiments described above should be interpreted as illustrative and not limited in any aspect.

### INDUSTRIAL APPLICABILITY

A pharmaceutical composition comprising squirrel fibroma virus and a reovirus, or a biological sample treated with the two types of viruses according to the present invention has the effect of inhibiting the growth of cancer cells by exhibiting oncolytic activity by specifically infecting cancer cells without affecting normal human cells.

In addition, since the combination of squirrel fibroma virus and a reovirus exhibits a very excellent synergistic effect compared to the individual viral therapy, the present invention can be effectively used in prevention and treatment of cancer.

1. A method of treating or alleviating cancer, comprising:
administering to a subject in need thereof a composition comprising an effective amount of (a) squirrel fibroma virus and a reovirus; or (b) a biological sample treated with the viruses of (a).

2. The method of claim 1, wherein the cancer is selected from the group consisting of gastric cancer, glioma, lung cancer, liver cancer, melanoma, prostate cancer, blood cancer, breast cancer, colorectal cancer, pancreatic cancer, brain cancer, ovarian cancer, and a combination thereof.

3. The method of claim 1, wherein the squirrel fibroma virus or reovirus comprises a heterologous gene for treating cancer.

4. The method of claim 3, wherein the heterologous gene for treating cancer is inserted into an essential region or non-essential region in a squirrel fibroma virus gene or reovirus gene to increase anticancer activity.

5. The method of claim 1, wherein the squirrel fibroma virus or reovirus is loaded in virus carrier cells and administered into the body.

6. The method of claim 1, wherein the squirrel fibroma virus is comprised at 10² to 10¹³ PFU.

7. The method of claim 1, wherein the reovirus is comprised at 10² to 10¹⁵ PFU.

8. The method of claim 1, wherein the titer ratio of the squirrel fibroma virus and the reovirus is 1:1 to 1000:1 (reovirus:squirrel fibroma virus).

9. The method of claim 1, wherein the biological sample is prepared by applying an effective amount of squirrel fibroma virus and reovirus to an ex vivo biological sample to kill a plurality of cancer cells.

10. The method of claim 1, wherein the biological sample is a bone marrow sample, an adipose-derived stem cell sample, or a blood sample.

11. The method of claim 1, wherein the squirrel fibroma virus is simultaneously, separately, or sequentially administered with the reovirus.

12. The method of claim 1, wherein the squirrel fibroma virus or reovirus is obtained using baby hamster kidney (BHK) cells.

13. The method of claim 1, wherein the pharmaceutical composition further comprises a chemotherapeutic or immunotherapeutic agent.

14.-15. (canceled)

16. The method of claim 6, wherein the reovirus is comprised at 10² to 10¹⁵ PFU.

17. The method of claim 9, wherein the biological sample is a bone marrow sample, an adipose-derived stem cell sample, or a blood sample.
